(19) United States
(12) Patent Application Publication
(10) Pub. No.: US 2011/0085978 A1
(43) Pub. Date: Apr. 14, 2011

(54) NOVEL QUINOLINYLAMIDE DERIVATIVES USEFUL AS MODULATORS OF DOPAMINE AND SEROTONIN RECEPTORS

(75) Inventors: Dan Peters, Malmo (SE); Lars Christian Rønn, Hillerod (DK); Karin Sandager Nielsen, Fredensborg (DK); Jørgen Scheel-Krüger, Glostrup (DK)

(73) Assignee: NeuroSearch A/S, Ballerup (DK)

(21) Appl. No.: 12/922,368

(22) PCT Filed: Mar. 13, 2009
(86) PCT No.: PCT/EP2009/052973

§ 371 Date: Dec. 8, 2010

Related U.S. Application Data
(60) Provisional application No. 61/036,674, filed on Mar. 14, 2008.

(30) Foreign Application Priority Data
Mar. 14, 2008 (DK) PA 2008 00396

(51) Int. Cl. A61K 49/00; C07D 401/12; A61K 31/496; C07D 401/14; A61K 31/4709; A61K 31/5513; A61P 25/18; A61P 25/00

(52) U.S. Cl. 424/9.1; 544/363; 514/253.06; 546/194; 514/318; 540/575; 514/218

(57) ABSTRACT

This invention provides novel quinolinylamide derivatives having medical utility, in particular as modulators of dopamine and serotonin receptors, preferably the D3, 5HT_{1A} and 5-HT_{2A} receptor subtypes, and in particular useful for the treatment of neuropsychiatric disorders, incl. schizophrenia.

### TECHNICAL FIELD

This invention provides novel quinolinylamide derivatives having medical utility, in particular as modulators of dopamine and serotonin receptors, preferably the D₃, 5HT_{1A} and 5-HT_{2A} receptor subtypes, and in particular useful for the treatment of neuropsychiatric disorders, incl. schizophrenia.

### BACKGROUND ART

Dopamine is involved in several important functions, excitatory and inhibitory, via dopaminergic receptors in the central and peripherical nervous system. Dopamine receptors were originally classified into two main groups: D₁ and D₂. The five currently cloned dopamine receptors fall into these classes. Thus, the D₂-like receptors include D₁ and D₅, while the D₂-like receptors include D₂, D₃ and D₄.

The dopamine receptors, and in particular the D₂-like receptors, are recognised as potential therapeutic targets for various neurological and psychiatric disorders, in particular psychotic disorders, incl. schizophrenia. Other therapeutic indications associated with the dopamine receptors include depression, Parkinson's disease, Huntington's disease, movement disorders such as dystonia, anxiety, restlessness, obsessive-compulsive disorders, mania, geriatric disorders, dementia, sexual dysfunction, musculo-skeletal pain symptoms, e.g. pain associated with fibromyalgia, substance abuse (cocaine abuse and addiction), abuse liability and withdrawal symptoms in drug addicts, and sleep disorders.

Still other therapeutic indications include eating disorders such as overeating, compulsive overeating, inability to regulate eating, bulimia and binge-eating disorder.

Also the compounds of the invention may be useful for the treatment of abuse liability and withdrawal symptoms caused by termination of use of addictive substances. Such addictive substances include nicotine containing products such as tobacco, opioids such as heroin, cocaine and morphine, cannabis, benzodiazepines, benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

Finally receptor selective ligands find use as diagnostic tools in diagnostic methods, and in particular for in vivo receptor imaging (neuroimaging).

WO 2006/072608 describes aryl piperazine derivatives useful as as modulators of dopamine and serotonin receptors.

WO 03/028728 describes certain substituted piperazinyl-butyl-carboxamides useful as dopamine D3 selective ligands.

Leopoldo et al. (J Med Chem 2002 45 (26) 5727-5735) describe a structure-affinity relationship study on certain N-[4-(4-arylpiperazin-1-yl)butyl]arylcarboxamides, useful as potent and selective dopamine D3 receptor ligands.

Campiani et al. (J Med Chem 2003 46 (18) 3822-3839) describe the synthesis and pharmacological evaluation of certain potent and highly selective D3 receptor ligands.

Hackling et al. (J Med Chem 2003 46 (18) 3883-3899) describe certain N-(omega-(4-(2-methoxyphenyl)piperazin-1-yl)alkyl)carboxamides useful as dopamine D2 and D3 receptor ligands.

U.S. Pat. No. 6,100,255 describes certain aryl aryl piperazine derivatives having activity at dopamine D4 receptor subtypes.

WO 2004/112729 describes certain aryl piperazine derivatives having activity at the D2 receptor.

WO 2004/033426 describes certain aryl piperazine and piperidine derivatives having activity at dopamine D2 receptor subtypes.

WO 96/02246 describes certain aryl piperazine and piperidine derivatives having activity at dopamine D3 receptor subtypes.

WO 2006/058993 describes certain aryl piperazine and piperidine derivatives having activity at dopamine D3 receptor subtypes.

U.S. Pat. No. 4,803,203 discloses certain heterocyclic piperazinyl alkoxy-benzheterocyclic derivatives useful as antipsychotic agents.

WO 94/22839 describes benzimidazole derivatives having affinity for the dopamine D4 receptor subtype.

WO 2004/024878 describes dopamine D3 receptor selective ligands.

WO 2004/004729 describes certain aryl piperazine and piperidine derivatives having activity at dopamine D3 receptor subtypes.

EP 409048 describes certain aryl piperazine and piperidine derivatives having activity at dopamine D2 receptor subtypes.

WO 01/49677 describes certain indolyl piperazine derivatives having activity at dopamine D4 receptor subtypes.

Leopoldo et al. (J Med Chem 2006 49 358-365) describe the design, synthesis, and binding affinities of potential PET ligands for visualization of brain dopamine D3 receptors.

However, the quinolinylamide derivatives of the present invention have not been reported.

### SUMMARY OF THE INVENTION

According to the present invention it has now been found that a particular group of quinolinylamide derivatives show superior activity as modulators of dopamine and serotonin receptors, preferably the D₃, 5HT_{1A} and 5-HT_{2A} receptor subtypes, has no significant activity on hERG, and has a good bioavailability when administered p.o.

Therefore, in its first aspect, the invention provides novel quinolinylamide derivatives represented by Formula I

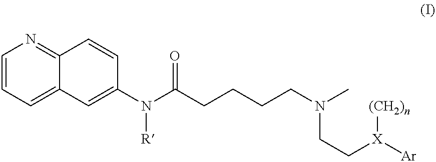

a stereoisomer thereof or a mixture of its stereoisomers, or an N-oxide thereof, or a pharmaceutically acceptable salt thereof, wherein

X represents CH or N;

n is 2 or 3;

R′ represents hydrogen or alkyl; and

Ar represents phenyl or piridinyl, which phenyl and pyridinyl are optionally substituted one or more times with substituents selected from alkyl, hydroxy, alkoxy, halo, trifluoromethyl, nitro and cyano.

In another aspect the invention relates to the use of the quinolinylamide derivative of the invention, or a pharmaceutically acceptable salt thereof, or a prodrug thereof for the manufacture of a pharmaceutical composition.

Viewed from yet another aspect the invention relates to the use of the quinolinylamide derivative of the invention, or a pharmaceutically acceptable salt thereof, or a prodrug thereof, for use as a medicament, or for the manufacture of a pharmaceutical composition for the treatment, prevention or alleviation of a disease or a disorder or a condition of a mammal, including a human, which disease, disorder or condition is responsive to modulation of the dopamine and serotonin receptors.

In a final aspect the invention provides a method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor subtypes, which method comprises the step of administering to such a living animal body in need thereof, a therapeutically effective amount of the quinolinylamide derivative of the invention, or a pharmaceutically acceptable salt thereof, or a prodrug thereof.

Other objects of the invention will be apparent to the person skilled in the art from the following detailed description and examples.

### DETAILED DISCLOSURE OF THE INVENTION

According to the present invention it has now been found that a particular group of quinolinylamide derivatives show a superior biological profile as modulators of dopamine and serotonin receptors.

Therefore, in its first aspect, the invention provides novel quinolinylamide derivatives represented by Formula I

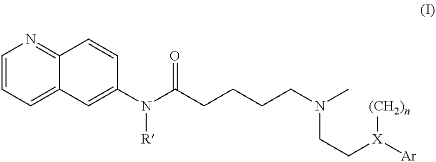

a stereoisomer thereof or a mixture of its stereoisomers, or an N-oxide thereof, or a pharmaceutically acceptable salt thereof, wherein

X represents CH or N;

n is 2 or 3;

R′ represents hydrogen or alkyl; and

Ar represents phenyl or piridinyl, which phenyl and pyridinyl are optionally substituted one or more times with substituents selected from alkyl, hydroxy, alkoxy, halo, trifluoromethyl, nitro and cyano.

In a more preferred embodiment the quinolinylamide derivative of the invention is a compound of Formula IA

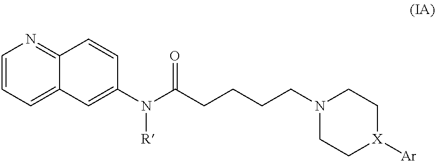

a stereoisomer thereof or a mixture of its stereoisomers, or an N-oxide thereof, or a pharmaceutically acceptable salt thereof, wherein

X represents CH or N;

R′ represents hydrogen or alkyl; and

Ar represents phenyl or piridinyl, which phenyl and pyridinyl are optionally substituted one or more times with substituents selected from alkyl, hydroxy, alkoxy, halo, trifluoromethyl, nitro and cyano.

In another more preferred embodiment the quinolinylamide derivative of the invention is a compound of Formula IB

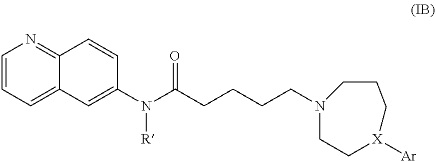

a stereoisomer thereof or a mixture of its stereoisomers, or an N-oxide thereof, or a pharmaceutically acceptable salt thereof, wherein

X represents CH or N;

R′ represents hydrogen or alkyl; and

Ar represents phenyl or pyridinyl, which phenyl and pyridinyl are optionally substituted one or more times with substituents selected from alkyl, hydroxy, alkoxy, halo, trifluoromethyl, nitro and cyano.

In a third more preferred embodiment the quinolinylamide derivative of the invention is a compound of Formula II

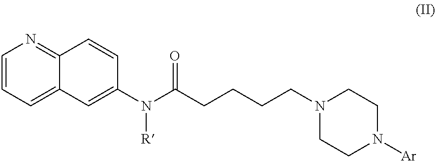

a stereoenantiomer thereof or a mixture of its stereoenantiomers, or a pharmaceutically acceptable salt thereof, wherein

R′ represents hydrogen or alkyl; and

Ar represents phenyl, optionally substituted one or more times with substituents selected from alkyl, alkoxy, halo, trifluoromethyl, nitro and cyano.

In a preferred embodiment the quinolinylamide derivative of the invention is a compound of Formula I, IA or IB, a stereoisomer thereof or a mixture of its stereoisomers, or an N-oxide thereof, or a pharmaceutically acceptable salt thereof,

wherein X represents CH or N.

In a more preferred embodiment X represents CH.

In another more preferred embodiment X represents N.

In another preferred embodiment the quinolinylamide derivative of the invention is a compound of Formula I, a stereoisomer thereof or a mixture of its stereoisomers, or an N-oxide thereof, or a pharmaceutically acceptable salt thereof, wherein n is 2 or 3.

In a more preferred embodiment n is 2.

In another more preferred embodiment n is 3.

In a third preferred embodiment the quinolinylamide derivative of the invention is a compound of Formula I, IA, IB or II, a stereoisomer thereof or a mixture of its stereoisomers, or an N-oxide thereof, or a pharmaceutically acceptable salt thereof, wherein R′ represents hydrogen or alkyl.

In a more preferred embodiment R′ represents hydrogen.

In another more preferred embodiment R′ represents alkyl, and in particular methyl or ethyl.

In a fourth preferred embodiment the quinolinylamide derivative of the invention is a compound of Formula I, IA, IB or II, a stereoisomer thereof or a mixture of its stereoisomers, or an N-oxide thereof, or a pharmaceutically acceptable salt thereof, wherein Ar represents phenyl or pyridinyl, which phenyl and pyridinyl are optionally substituted one or two times with substituents selected from alkyl, hydroxy, alkoxy, halo, trifluoromethyl, nitro and cyano.

In a more preferred embodiment Ar represents phenyl or pyridinyl, which phenyl and pyridinyl are optionally substituted one or two times with substituents selected from alkyl and alkoxy.

In another more preferred embodiment Ar represents phenyl, optionally substituted with alkoxy, and in particular methoxy.

In a third more preferred embodiment Ar represents pyridinyl, optionally substituted with alkyl, and in particular methyl.

In a fourth more preferred embodiment Ar represents phenyl, optionally substituted one or more times with substituents selected from alkyl, alkoxy, halo, trifluoromethyl, nitro and cyano.

In a fifth more preferred embodiment Ar represents phenyl, optionally substituted one or two times with substituents selected from alkyl, alkoxy, halo, trifluoromethyl, nitro and cyano.

In a sixth more preferred embodiment Ar represents phenyl, optionally substituted one or two times with substituents selected from alkyl, alkoxy, halo, trifluoromethyl, nitro and cyano.

In a seventh more preferred embodiment Ar represents phenyl, optionally substituted one or two times with substituents selected from halo and trifluoromethyl.

In an eight more preferred embodiment Ar represents phenyl.

In a most preferred embodiment the quinolinylamide derivative of the invention is
5-(4-Phenyl-piperazin-1-yl)-pentanoic acid quinolin-6-ylamide;
5-(4-Phenyl-piperazin-1-yl)pentanoic acid methyl-quinolin-6-yl-amide;
5-[4-(6-Methyl-pyridin-2-yl)piperazin-1-yl]-pentanoic acid quinolin-6-ylamide;
5-[4-(6-Methyl-pyridin-2-yl)piperazin-1-yl]-pentanoic acid methyl-quinolin-6-yl-amide;
5-(4-Phenyl-piperazin-1-yl)pentanoic acid ethyl-quinolin-6-yl-amide;
5-[4-(2-Methoxy-phenyl)piperazin-1-yl]-pentanoic acid methyl-quinolin-6-yl-amide;
5-[4-(2-Methoxy-phenyl)piperazin-1-yl]-pentanoic acid quinolin-6-ylamide;
5-(1-Oxy-4-phenyl-piperazin-1-yl)pentanoic acid quinolin-6-ylamide;
5-(4-Phenyl-piperidin-1-yl)pentanoic acid quinolin-6-ylamide;
5-(4-Phenyl-piperidin-1-yl)pentanoic acid methyl-quinolin-6-yl-amide;
5-(4-Phenyl-[1,4]diazepan-1-yl)pentanoic acid quinolin-6-ylamide; or
5-(4-Phenyl-[1,4]diazepan-1-yl)pentanoic acid methyl-quinolin-6-yl-amide;

a stereoisomer thereof or a mixture of its stereoisomers, or an N-oxide thereof, or a pharmaceutically acceptable salt thereof.

Any combination of two or more of the embodiments described herein is considered within the scope of the present invention.

### Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy, ethoxy and isopropoxy.

In the context of this invention halo represents fluoro, chloro, bromo or iodo.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. A stereo-selective synthetic approach may be pursued. Racemic compounds of the present invention can thus be resolved into their optical antipodes, e.g., by fractional crystallisation of D- or L- (tartrates, mandelates or camphorsulphonate) salts for example.

Starting materials and/or intermediate compounds used for producing the chemical compounds of the present invention may also be resolved by the formation of diastereomeric amides by reaction of the quinolinylamide derivative of the present invention with an optically active activated carboxylic acid such as that derived from (+) or (−) phenylalanine, (+) or (−) phenylglycine, (+) or (−) camphanic acid or by the formation of diastereomeric carbamates by reaction of the starting material or intermediate compound for use according to the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Pharmaceutically Acceptable Salts

The quinolinylamide derivatives of the invention may be provided in any form suitable for the intended administration. Suitable forms include pharmaceutically (i.e. physiologically) acceptable salts, and pre- or prodrug forms of the quinolinylamide derivatives of the invention.

Examples of pharmaceutically acceptable salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

### Methods of Preparation

The quinolinylamide derivatives of the invention may be prepared by conventional methods for chemical synthesis, e.g. those described in the working examples.

Intermediate compounds invention may be resolved by the formation of diastereomeric amides by reaction with an optically active activated carboxylic acid such as that derived from (+) or (−) phenylalanine, (+) or (−) phenylglycine, (+) or (−) camphanic acid or by the formation of diastereomeric carbamates by reaction of the intermediate compound with an optically active chloroformate or the like.

### Biological Activity

The quinolinylamide derivatives of the invention were found to possess selectivity for the dopamine and serotonin receptors. Therefore, in a preferred embodiment, the invention relates to use of the quinolinylamide derivatives of the invention for the treatment, prevention or alleviation of a disease or a disorder or a condition of a mammal, including a human, which disease, disorder or condition is responsive to modulation of the dopamine and serotonin receptors, in particular the D₃, 5HT_{1A} and 5-HT_{2A} receptor subtypes.

Moreover, the quinolinylamide derivatives of the invention has no significant activity on hERG, and has a good bioavailability when administered p.o.

Therefore, in a preferred embodiment, the invention relates to use of the quinolinylamide derivatives of the invention for the treatment, prevention or alleviation of a disease or a disorder or a condition of a mammal, including a human, which disease, disorder or condition is responsive to modulation of the dopamine and serotonin receptors.

In a more preferred embodiment the disease, disorder or condition is a neurological or psychiatric disorders, in particular psychotic disorders, incl. schizophrenia, depression, Parkinson's disease, Huntington's disease, movement disorders, in particular dystonia, anxiety, restlessness, obsessive-compulsive disorders, mania, geriatric disorders, dementia, sexual dysfunction, musculo-skeletal pain symptoms, in particular pain associated with fibromyalgia, sleep disorders, substance abuse or addiction, and abuse liability and withdrawal symptoms in drug addicts, cocaine abuse or addiction.

In an even more preferred embodiment the disease, disorder or condition is a neurological or psychiatric disorder, in particular a psychotic disorder, preferably schizophrenia.

In another preferred embodiment the disease, disorder or condition contemplated according to the invention is schizophrenia or Parkinson's disease.

In a third preferred embodiment the disease, disorder or condition contemplated according to the invention an eating disorder, overeating, compulsive overeating, inability to regulate eating, bulimia or binge-eating disorder.

In a fourth preferred embodiment the disease, disorder or condition contemplated according to the invention is abuse liability or withdrawal symptoms caused by termination of use of addictive substances. Such addictive substances include nicotine containing products such as tobacco, opioids such as heroin, cocaine and morphine, cannabis, benzodiazepines, benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In yet another preferred embodiment the quinolinylamide derivatives of the invention are used as diagnostic tools in diagnostic methods, and in particular for in vivo receptor imaging (neuroimaging).

### Pharmaceutical Compositions

In another aspect the invention provides novel pharmaceutical compositions comprising a therapeutically effective amount of the quinolinylamide derivative of the invention.

While an quinolinylamide derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the quinolinylamide derivative of the invention, or a pharmaceutically acceptable salt or derivative thereof, together with one or more pharmaceutically acceptable carriers, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the diagnosis, treatment, prevention or alleviation of a disease or a disorder or a condition of a living animal body, including a human, which disease, disorder or condition is responsive to modulation of the dopamine and serotonin receptors, and which method comprises administering to such a living animal body, including a human, in need thereof an effective amount of an quinolinylamide derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Preparatory Example

### Method A

### 5-(4-Phenyl-piperazin-1-yl)-pentanoic acid quinolin-6-ylamide fumaric acid salt (Compound A1)

A mixture of 5-bromo-pentanoic acid quinolin-6-ylamide (1.0 g, 3.26 mmol), 1-phenylpiperazine (0.53 g, 3.26 mmol), triethylamine (0.36 g, 3.58 mmol) and acetonitrile (35 ml) was stirred at reflux for 15 h. Aqueous sodium hydroxide (30 ml, 1 M) was added and the mixture was extracted with dichloromethane (50 ml). Chromatography on silica gel with dichloromethane, 10% methanol and 1% aqueous ammonia as solvent gave the product. Yield 850 mg (67%). The corresponding salt was obtained by addition of a diethyl ether and methanol mixture (9:1) saturated with fumaric acid. LC-ESI-HRMS of [M+H]+ shows 389.2321 Da. Calc. 389.234136 Da, dev. −5.2 ppm.

### 5-Bromo-pentanoic acid quinolin-6-ylamide (Intermediate compound)

A mixture of 5-bromovaleryl chloride (4.03 g, 19.61 mmol) and dichloromethane (30 ml) was cooled to 0° C. 6-Aminoquinoline (2.57 g, 17.8 mmol) was added in small portions during 15 minutes. The temperature was allowed to reach room-temperature and stir for 15 h. Aqueous sodium hydroxide (40 ml, 1 M) was added and the mixture was extracted with dichloromethane (60 ml). The mixture was evaporated and diethylether (5 ml) was added, the solid was filtered and washed with diethylether. Yield 4.95 g (90%).

### 5-[4-(6-Methyl-pyridin-2-yl)-piperazin-1-yl]-pentanoic acid quinolin-6-ylamide (Compound A2)

Was prepared according to Method A. LC-ESI-HRMS of [M+H]+ shows 404.2446 Da. Calc. 404.24449 Da, dev. 0.3 ppm.

### 5-[4-(2-Methoxy-phenyl)-piperazin-1-yl]-pentanoic acid quinolin-6-ylamide (Compound A3)

Was prepared according to Method A. LC-ESI-HRMS of [M+H]+ shows 419.2435 Da. Calc. 419.244156 Da, dev. −1.6 ppm.

### 5-(4-Phenyl-piperidin-1-yl)-pentanoic acid quinolin-6-ylamide (Compound A4)

Was prepared according to Method A. LC-ESI-HRMS of [M+H]+ shows 388.2397 Da. Calc. 388.238342 Da, dev. 3.5 ppm.

### 5-(4-Phenyl-[1,4]diazepan-1-yl)-pentanoic acid quinolin-6-ylamide (Compound A5)

Was prepared according to Method A. LC-ESI-HRMS of [M+H]+ shows 403.2492 Da. Calc. 403.249241 Da, dev. −0.1 ppm.

### 5-(1-Oxy-4-phenyl-piperazin-1-yl)-pentanoic acid quinolin-6-ylamide (Compound A6)

Was prepared by oxidation of 5-(4-Phenyl-piperazin-1-yl)-pentanoic acid quinolin-6-ylamide (Compound I). LC-ESI-HRMS of [M+H]+ shows 405.2282 Da. Calc. 405.228506 Da, dev. −0.8 ppm.

### 5-Bromo-pentanoic acid quinolin-6-ylamide (Intermediate compound)

A mixture of 5-bromovaleryl chloride (4.03 g, 19.61 mmol) and dichloromethane (30 ml) was cooled to 0° C. 6-Aminoquinoline (2.57 g, 17.8 mmol) was added in small portions during 15 minutes. The temperature was allowed to reach room-temperature and stir for 15 h. Aqueous sodium hydroxide (40 ml, 1 M) was added and the mixture was extracted with dichloromethane (60 ml). The mixture was evaporated and diethylether (5 ml) was added, the solid was filtered and washed with diethylether. Yield 4.95 g (90%).

### Method B

### 5-(4-Phenyl-piperazin-1-yl)-pentanoic acid methyl-quinolin-6-yl-amide fumaric acid salt (Compound B1)

5-(4-Phenyl-piperazin-1-yl)-pentanoic acid quinolin-6-ylamide (Compound 1; 0.425 g, 1.09 mmol) and sodium tert-butoxide (0.115 g 1.20 mmol) was solved in THF (20 ml) and cooled to 0° C. Iodomethane (0.155 g, 1.09 mmol) was added dropwise at 0° C. Aqueous sodium hydroxide (10 ml, 1 M) was added and the mixture was extracted with dichloromethane (20 ml). Chromatography on silica gel with dichloromethane, 10% methanol and 1% aqueous ammonia as solvent gave the product. Yield 128 mg (29%). The corresponding salt was obtained by addition of a diethyl ether and methanol mixture (9:1) saturated with fumaric acid. LC-ESI-HRMS of [M+H]+ shows 403.2507 Da. Calc. 403.249786 Da, dev. 2.3 ppm.

### 5-[4-(6-Methyl-pyridin-2-yl)-piperazin-1-yl]-pentanoic acid methyl-quinolin-6-yl-amide (Compound B2)

Was prepared according to Method B. LC-ESI-HRMS of [M+H]+ shows 418.2607 Da. Calc. 418.26014 Da, dev. 1.3 ppm.

### 5-(4-Phenyl-piperazin-1-yl)-pentanoic acid ethyl-quinolin-6-yl-amide (Compound B3)

Was prepared according to Method B using iodoethane at 50° C. LC-ESI-HRMS of [M+H]+ shows 417.2638 Da. Calc. 417.265436 Da, dev. −3.9 ppm.

### 5-[4-(2-Methoxy-phenyl)-piperazin-1-yl]-pentanoic acid methyl-quinolin-6-yl-amide (Compound B4)

Was prepared according to Method B. LC-ESI-HRMS of [M+H]+ shows 433.2612 Da. Calc. 433.259806 Da, dev. 3.2 ppm.

### 5-(4-Phenyl-piperidin-1-yl)-pentanoic acid methyl-quinolin-6-yl-amide (Compound B5)

Was prepared according to Method B. LC-ESI-HRMS of [M+H]+ shows 402.2546 Da. Calc. 402.253992 Da, dev. 1.5 ppm.

### 5-(4-Phenyl-[1,4]diazepan-1-yl)-pentanoic acid methyl-quinolin-6-yl-amide (Compound B6)

Was prepared according to Method B. LC-ESI-HRMS of [M+H]+ shows 417.2646 Da. Calc. 417.264891 Da, dev. −0.7 ppm.

### Example 2

### Biological Activity

### In Vitro Binding Studies

In this experiment compounds representative of the invention are being screened for affinity towards various receptors (e.g. D2, D3, 5ht1a, 5ht2a) in routine radioligand binding assays (MDS).

### In Vivo Profiling

Female NMRI mice (Taconic M&B, P.O. box 1079, DK-8680 Ry, Denmark) are kept in a ventilated closed rack (Scantainer, Scanbur Ltd., Denmark) at constant temperature (21° C.) and humidity (60-70%) with a 7:00 a.m light/7:00 p.m dark cycle. Food (altromin rat pellets) and water were available ad libitum.

Experiments were conducted during daytime in the light phase. The animals were habituated to the experimental room approximately 24 hours before start of the experiments.

### MK801 Induced Hyperactivity

MK801 induced hyperactivity was measured in automated activity frames (TSE Home Cage Activity Monitoring System, MoTil, TSE technical & scientific equipment GmbH, Germany) equipped with infrared photobeam emitters and sensors. MK801 was administered in a dose of 0.2 mg/kg ip immediately before test start.

Test compound was pretreated subcutaneously 30 minutes before test start at the following doses 3, 10, 30 mg/kg in a dose volume of 10 ml/kg.

Data are reported as the Minimal Effective Dose (M.E.D) that significantly reduces MK801 induced hyperactivity.

| Test compound | M.E.D (mg/kg) |
|---|---|
| Compound A3 | 10 |
| Compound A4 | 10 |

1. A quinolinylamide derivative represented by Formula 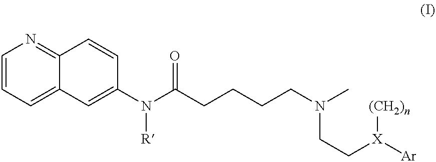
a stereoisomer thereof or a mixture of its stereoisomers, or an N-oxide thereof, or a pharmaceutically acceptable salt thereof, wherein
X represents CH or N;
n is 2 or 3;
R′ represents hydrogen or alkyl; and
Ar represents phenyl or pyridinyl, which phenyl and pyridinyl are optionally substituted one or more times with substituents selected from alkyl, hydroxy, alkoxy, halo, trifluoromethyl, nitro and cyano.

2. The quinolinylamide derivative of claim 1, a stereoisomer thereof or a mixture of its stereoisomers, or an N-oxide thereof, or a pharmaceutically acceptable salt thereof, wherein X represents CH or N.

3. The quinolinylamide derivative of claim 1, a stereoisomer thereof or a mixture of its stereoisomers, or an N-oxide thereof, or a pharmaceutically acceptable salt thereof, wherein n is 2 or 3.

4. The quinolinylamide derivative of claim 1, a stereoisomer thereof or a mixture of its stereoisomers, or an N-oxide thereof, or a pharmaceutically acceptable salt thereof, wherein R′ represents hydrogen or alkyl.

5. The quinolinylamide derivative of claim 1, a stereoisomer thereof or a mixture of its stereoisomers, or an N-oxide thereof, or a pharmaceutically acceptable salt thereof, wherein Ar represents phenyl or pyridinyl, which phenyl and pyridinyl are optionally substituted one or two times with substituents selected from alkyl, hydroxy, alkoxy, halo, trifluoromethyl, nitro and cyano.

6. The quinolinylamide derivative of claim 1, which is
5-(4-Phenyl-piperazin-1-yl)-pentanoic acid quinolin-6-ylamide;
5-(4-Phenyl-piperazin-1-yl)-pentanoic acid methyl-quinolin-6-yl-amide;
5-[4-(6-Methyl-pyridin-2-yl)-piperazin-1-yl]-pentanoic acid quinolin-6-ylamide;
5-[4-(6-Methyl-pyridin-2-yl)-piperazin-1-yl]-pentanoic acid methyl-quinolin-6-yl-amide;
5-(4-Phenyl-piperazin-1-yl)-pentanoic acid ethyl-quinolin-6-yl-amide;
5-[4-(2-Methoxy-phenyl)-piperazin-1-yl]-pentanoic acid methyl-quinolin-6-yl-amide;
5-[4-(2-Methoxy-phenyl)-piperazin-1-yl]-pentanoic acid quinolin-6-ylamide;
5-(1-Oxy-4-phenyl-piperazin-1-yl)-pentanoic acid quinolin-6-ylamide;
5-(4-Phenyl-piperidin-1-yl)-pentanoic acid quinolin-6-ylamide;
5-(4-Phenyl-piperidin-1-yl)-pentanoic acid methyl-quinolin-6-yl-amide;
5-(4-Phenyl-[1,4]diazepan-1-yl)-pentanoic acid quinolin-6-ylamide; or
5-(4-Phenyl-[1,4]diazepan-1-yl)-pentanoic acid methyl-quinolin-6-yl-amide;
a stereoisomer thereof or a mixture of its stereoisomers, or an N-oxide thereof, or a pharmaceutically acceptable salt thereof.

7. A pharmaceutical composition comprising a therapeutically effective amount of a quinolinylamide derivative of claim 1, a stereoisomer thereof or a mixture of its stereoisomers, or an N-oxide thereof, or a pharmaceutically acceptable addition salt thereof, together with at least one pharmaceutically acceptable carrier or diluent.

8. (canceled)

9. (canceled)

10. (canceled)

11. (canceled)

12. (canceled)

13. A method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{IA} or D₃/5-HT_{2A} receptor subtypes, which method comprises the step of administering to such a living animal body in need thereof, a therapeutically effective amount of a quinolinylamide derivative according to claim 1, a stereoisomer thereof or a mixture of its stereoisomers, or an N-oxide thereof, a pharmaceutically acceptable salt thereof.

14. The method according to claim 13, wherein the disease or a disorder or a condition is a neurological or psychiatric disorders, in particular psychotic disorders, schizophrenia, depression, Parkinson's disease, Huntington's disease, movement disorders, dystonia, anxiety, restlessness, obsessive-compulsive disorders, mania, geriatric disorders, dementia, sexual dysfunction, musculo-skeletal pain symptoms, pain associated with fibromyalgia, sleep disorders, substance abuse or addiction, and abuse liability and withdrawal symptoms in drug addicts, cocaine abuse or addiction.

15. The method according to claim 13, wherein the disease or a disorder or a condition is a neurological or psychiatric disorder, in particular a psychotic disorder, preferably schizophrenia.
